(19) United States
(12) Patent Application Publication
(10) Pub. No.: US 2025/0188403 A1
(43) Pub. Date: Jun. 12, 2025

(54) CLOSED CELL SEPARATION OPERATION APPARATUS

(71) Applicant: DUOGENIC STEMCELLS CORPORATION, TAICHUNG CITY (TW)

(72) Inventors: HONG-LIN SU, TAICHUNG CITY (TW); CHING-I SHEN, TAICHUNG CITY (TW); FU-HUI WANG, TAICHUNG CITY (TW); CHIA-YING HSIEH, TAICHUNG CITY (TW)

(73) Assignee: DUOGENIC STEMCELLS CORPORATION, TAICHUNG CITY (TW)

(21) Appl. No.: 18/844,659

(22) PCT Filed: Mar. 22, 2022
(86) PCT No.: PCT/CN2022/082170

§ 371 Date: Sep. 6, 2024

(51) Int. Cl. C12M 1/00; C12M 1/12; C12M 1/26

(52) U.S. Cl. CPC C12M 47/04; C12M 23/06; C12M 23/38; C12M 29/20; C12M 33/10; C12M 33/14; C12M 33/22

(57) ABSTRACT

A closed cell separation operation apparatus includes a first tube body having a vent and an inlet, a second tube body having a vent and an inlet and a connecting pipe, connecting the first tube body and a second tube body; the vent of the first tube body allows air to enter and exit the first tube body and the inlet of the first body allows a fluid from an external fluid source to enter the first tube body; and the first tube body having an outlet; the vent of the second tube body allows air to enter and exit the second tube body and the inlet of the second tube body allows at least one part of the fluid from the outlet of the first tube body to enter the second tube body and the second tube body having an outlet. The closed cell separation operation apparatus provides a closed, contamination-free environment for both centrifugation and post-centrifugation supernatant separation.

### BACKGROUND OF THE INVENTION

### 1. Field of the Invention

The present invention relates to centrifugal separation, and more particularly to a closed cell separation operation apparatus with a design that provides a closed, contamination-free environment for both centrifugation and post-centrifugation supernatant separation. The closed cell separation operation apparatus is particularly useful in, for example, cell separation and isolation.

### 2. Description of the Related Art

Centrifugation has been extensively used in cell-related studies and assays to sort cells in such as bone marrow aspirate concentrate (BMAC), cerebrospinal fluid (CSF), peripheral blood, urine, ascites, cell culture media, etc.

As an essential tool for centrifugal separation, a conventional centrifuge tube is made as a capped test tube. Examples may be those traded under the name of Nunc (Thermo Fisher Scientific, the US). In use of such an existing centrifuge tube, a fluid to be subject to centrifugal processing is transferred into it, and then the centrifuge tube is sealed and placed into a centrifuge for processing. After centrifugation, for removing the resulting supernatant (e.g., plasma for whole blood centrifugation) and harvesting the sediment (e.g., blood cells for whole blood centrifugation), or vice versa, an operator has to separate the two parts by unsealing the tube and transferring the supernatant to a spear vessel. However, this operation unavoidably exposes the supernatant and sediment to the ambient environment and brings about risks of contamination. One solution to address the concern is to perform the whole operation in a clean room or a biosafety cabinet (BSC), which is usually costly and relatively immobile, thus unsuitable for clinical use.

### SUMMARY OF THE INVENTION

### Technical Problem

Another problem of the conventional practice for post-centrifugation supernatant separation is that spill tends to happen in the process of transferring the content of the centrifuge tube and reduce the volume of the available specimen. This may have adverse effects on the intended study or assay, or in the worst case, makes it impossible to perform the intended study or assay.

Hence, there is a need for a novel apparatus that allows post-centrifugation supernatant separation to be performed efficiently and effusively in a contamination-free manner while eliminating the risks of spill and the needs for costly laboratory equipment.

### Solution to Problems

### Technical Solution

In view of the aforementioned need, the primary objective of the present invention is to provide a closed cell separation operation apparatus that is designed to provide a closed, contamination-free environment for both centrifugation and post-centrifugation supernatant separation while eliminating the risks of spills and the needs for costly laboratory equipment.

In order to achieve this and other objectives of the present invention, the disclosed closed cell separation operation apparatus comprises: a first tube body, being a cylinder-like container defined by a peripheral wall, the first tube body having a closed top that sealably communicates with the exterior through a vent and an inlet, wherein the vent is for allowing an air to pass and enter or exit the first tube body and the inlet is for allowing a fluid from an external fluid source to enter the first tube body; and the first tube body having a closed bottom that sealably communicates with the exterior through a centrally formed outlet; a second tube body, being a cylinder-like container defined by a peripheral wall, the second tube body having a closed top that sealably communicates with the exterior through a vent and an inlet, wherein the vent is for allowing an air to pass and enter or exit the second tube body and the inlet is for allowing at least one part of the fluid from the outlet of the first tube body to enter the second tube body and the second tube body having a closed bottom that sealably communicates with the exterior through a centrally formed outlet; and a connecting pipe, being arranged between the outlet of the first tube body and the inlet of the second tube so as to connect the first tube body and a second tube body.

Further, the peripheral walls of the first tube body and the second tube body each extend downward beyond the bottom so the peripheral wall and the bottom form a pipe-receiving space around the outlet, and at least one of the connecting pipe and an extension pipe is configured to be at least partially coiled around the outlet of the tube body and received in the pipe-receiving space of tube body.

Further, in the closed cell separation operation apparatus, there is further an extension pipe attached to the outlet of the second tube body for connecting the second tube body to a further said first tube body of a further said closed cell separation operation apparatus.

Further, in the closed cell separation operation apparatus, at least one of the peripheral walls of the first tube body and the second tube body has a lower edge formed with a notch for the connecting pipe to pass therethrough, so that when the first tube body and the second tube body stands on a plane, an access to the pipe-receiving space of the first tube body and the second tube body is left for the connecting pipe to pass therethrough.

Further, in the closed cell separation operation apparatus, at least one of the connecting pipes and the extension pipe is equipped with a valve for closing or opening the pipe, and the valve is a tube clamp, a flow control valve, a multi-way tap connector or an on-off fluid valve.

Further, in the closed cell separation operation apparatus, wherein at least one of the bottoms of the first tube body and the second tube body has a downward conical structure with a diameter gradually reduces and reaches a smallest extent thereof at a periphery of the outlet of the first tube body and the second tube body.

Further, in the closed cell separation operation apparatus, at least one of the vents of the first tube body and the second tube body is equipped with a filter for filtering the air passing through the vent.

Further, in the closed cell separation operation apparatus, there is further an air-pressure source attached to at least one of the vents of the first tube body and the second tube body for pushing air into or drawing air from the first tube body and the second tube body.

The air-pressure source is an air pump or an air injector.

Further, in the closed cell separation operation apparatus, wherein at least one of the inlets and the vents of the first tube body and the second tube body is configured to be detachably sealed by a seal in an airtight manner.

Further, in the closed cell separation operation apparatus, at least one of the inlets and the vents of the first tube body and the second tube body is further provided with a pipe that is equipped with a vale.

The valve is a tube clamp, a flow control valve, a multi-way tap connector or an on-off fluid valve.

### BENEFICIAL EFFECTS OF THE INVENTION

### Beneficial Effects

The beneficial effects of the present invention are as follows:

The closed cell separation operation apparatus of the present invention provides a closed, contamination-free environment for both centrifugation and post-centrifugation supernatant separation while eliminating the risks of spills and the needs for costly laboratory equipment.

### BRIEF DESCRIPTION OF THE DRAWINGS

FIG. 1 is a perspective view of a closed cell separation operation apparatus according to one embodiment of the present invention;

FIG. 2 through FIG. 4 are applied, cross-sectional view of the closed cell separation operation apparatus of the present invention, illustrating the operation of the closed cell separation operation apparatus during centrifugation and post-centrifugation supernatant separation;

FIG. 3 is a schematic view of the following operation system, illustrating the vehicle body moving in the manual mode.

FIG. 5 is a perspective view of an alternative implementation of the closed cell separation operation apparatus, wherein two closed cell separation operation apparatus of the present invention are connected in series; and

FIG. 6 is a perspective view of a closed cell separation operation apparatus according to another embodiment of the present invention.

In the figures, 10: first tube body; 11,21: opening; 121,221: outlet; 12,22: bottom; 122, 222: peripheral wall; 123, 223: pipe-receiving space; 124, 224: notch; 13: first cap; 131, 231: vent; 132, 232: inlet; 20: second tube body; 23: second cap; 24: connecting pipe; 30: venting pipe; 31: filter; 40: feeding pipe; 50: extension pipe; 60: valve; 70: air-pressure source; 80: seal; 90: venting pipe; 91: additional filter.

### IMPLEMENTING THE PREFERRED EMBODIMENTS OF THE INVENTION

### Preferred Embodiments of the Invention

While a preferred embodiment provided hereinafter for illustrating the concept of the present invention has been described above, it is to be understood that the components of the embodiment shown in the accompanying drawings are depicted for the sake of easy explanation and need not to be made to exact scale.

For the descriptions herein, the singular forms “a”, and “an” include plural referents unless the context clearly indicates otherwise.

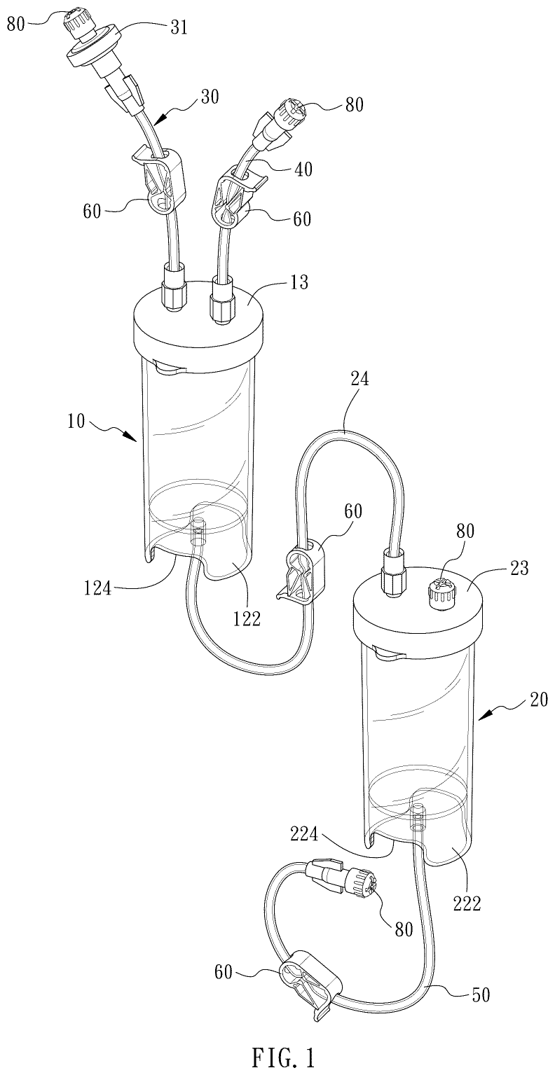

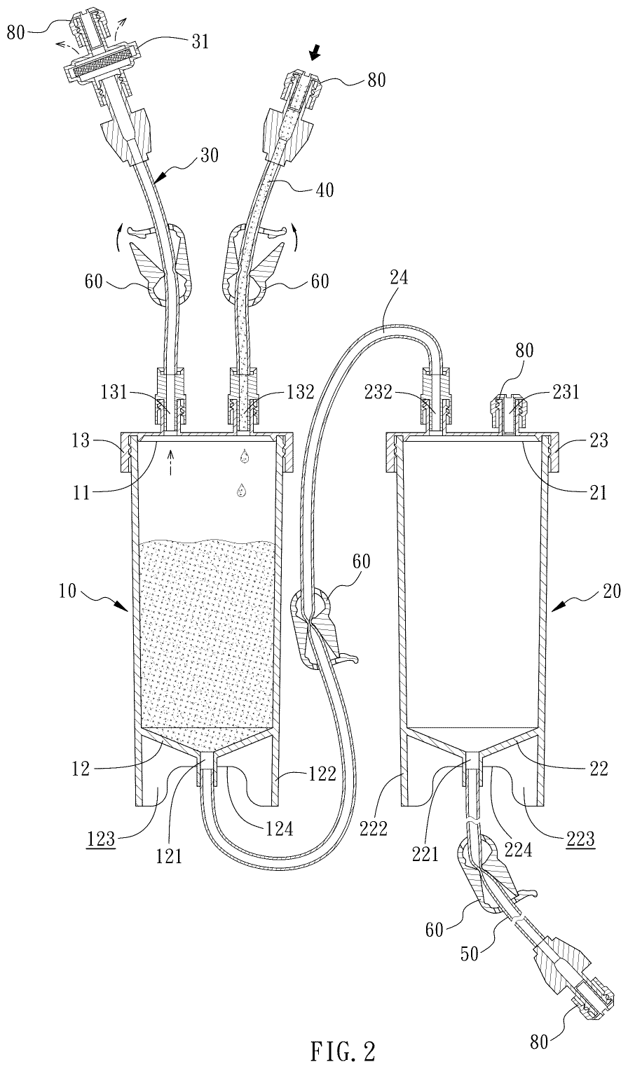

Referring first to FIG. 1 and FIG. 2, according to one embodiment of the present invention, a closed cell separation operation apparatus primarily comprises a first tube body 10 capped by a first cap 13, a second tube body 20 capped by a second cap 23, and a connecting pipe 24 connecting the two tube bodies 10, 20.

As the first tube body 10 and the second tube body 20 are substantively identical in terms of structure, the following description is directed to only one of them for succinctness. The first tube body 10 or the second tube body 20 is a cylinder-like container defined by a peripheral wall 122 or 222. The first tube body 10 or the second tube body 20 has an open top fully open to the exterior through an opening 11 or 21 and a closed bottom 12 or 22 that sealably communicates with the exterior through a centrally formed outlet 121 or 221. Particularly, the bottom 12 or 22 has a funnel-like, or downward conical structure with a diameter that gradually reduces and reaches its smallest extent at the periphery of the outlet 121 or 221. The peripheral wall 122 or 222 of the tube body 10 or 20 extends downward beyond the closed bottom 12 or 22 so that it works with the funnel-like bottom 12 or 22 to form a pipe-receiving space 123 or 223 around the outlet 121 or 221. The lower edge of the peripheral wall 122 or 222 is further formed with a notch 124 or 224, so that when the first tube body 10 or the second tube body 20 stands on a plane, an access to the pipe-receiving space 123 or 223 is created. It is to be noted that the first tube body 10 or the second tube body 20 is sized similar to the existing centrifuge tubes or blood collection tubes, so as to be compatible with existing centrifuges.

Similarly, since the first cap 13, a second cap 23 are substantively identical in terms of structure, the following description is directed to only one for succinctness. The first cap 13 or the second cap 23 is configured to cap the first tube body 10 or the second tube body 20 from top and has a vent 131 or 231 and an inlet 132 or 232. As detailed below, each of the vent 131 or 231 and the inlet 132 or 232 is configured to be removably connected to pipes in used and/or detachably capped by a seal 80 in an airtight manner when not in use.

It is to be understood that while the present embodiment has the first tube body 10 or the second tube body 20 capped by the cap 13 or 23 formed with the vent 131 or 231 and the inlet 132 or 232, the disclosed closed cell separation operation apparatus in another embodiment may have the vent 131 or 231 and the inlet 132 or 232 directly formed on a closed top surface of the first tube body 10 or the second tube body 20 instead of using the cap 13 or 23 to close the first tube body 10 or the second tube body 20.

The connecting pipe 24 is arranged between the outlet 121 of the first tube body 10 and the inlet 232 of the second tube body 20 to connect the first tube body 10 and the second tube body 20 for liquid communication. The connecting pipe 24 can be partially coiled around the outlet 121 of the first tube body 10 to be well received in the pipe-receiving space 123 of the first tube body 10. With the notch 124 formed on the peripheral wall 122 of the first tube body 10, the connecting pipe 24 can extend between the first tube body 10 and the second tube body 20 without preventing the first tube body 10 from standing on any plane stably. The connecting pipe 24 is further equipped with a valve 60 for closing or opening the connecting pipe 24.

In use, with the connecting pipe 24 closed by the valve 60, a feeding pipe 40 communicated with a source of a fluid to be processed (not shown), such as a filled blood bag, a blood gas syringe or a specimen tube, is connected to the inlet 132 of the first tube body 10. At this time, the vent 131 of the first tube body 10 remains open, so that the fluid to be processed (e.g., whole blood) can be transferred to the first tube body 10, as shown in FIG. 2. Herein, the vent 131 of the first tube body 10 may be further provide with a filter 31 for preventing foreign objects from entering the of the first tube body 10 and thereby ensuring a contamination-free operation environment. Specifically, as shown in FIG. 2, a venting pipe 30 equipped with a filter 31 is attached to the vent 131 of the first tube body 10. The venting pipe 30 may be also opened or released by a valve 60. After the fluid is filled into the first tube body 10, the inlet 132 of the first tube body 10 can be capped using a seal 80 directly in an airtight manner, or the feeding pipe 40 remained connected with the inlet 132 of the first tube body 10 is capped using a seal 80 in an airtight manner, as shown FIG. 1. Similarly, the vent 131 of the first tube body 10 can be capped using a seal 80 directly in an airtight manner, or the venting pipe 30 remained connected with the vent 131 is capped using a seal 80 in an airtight manner, as shown FIG. 1.

It is to be noted that when the connecting pipe 24, the venting pipe 30, and the feeding pipe 40 are closed by their respective valves 60, the interior of the first tube body 10 is sealed from the exterior and forms a closed environment. Also, the second tube body 20 is now with its vent 231 and outlet 221 sealed and isolated from the exterior.

Then the entire closed cell separation operation apparatus can be moved into a centrifuge (not shown) for centrifugation. Therein, the first tube body 10 and the second tube body 20 are seated in two adjacent places in the centrifuge, with the connecting pipe 24 partially coiled around the outlet 121 and received in the pipe-receiving space 123 of the first tube body 10. This prevents the connecting pipe 24 from undesired excessive movement during centrifugation.

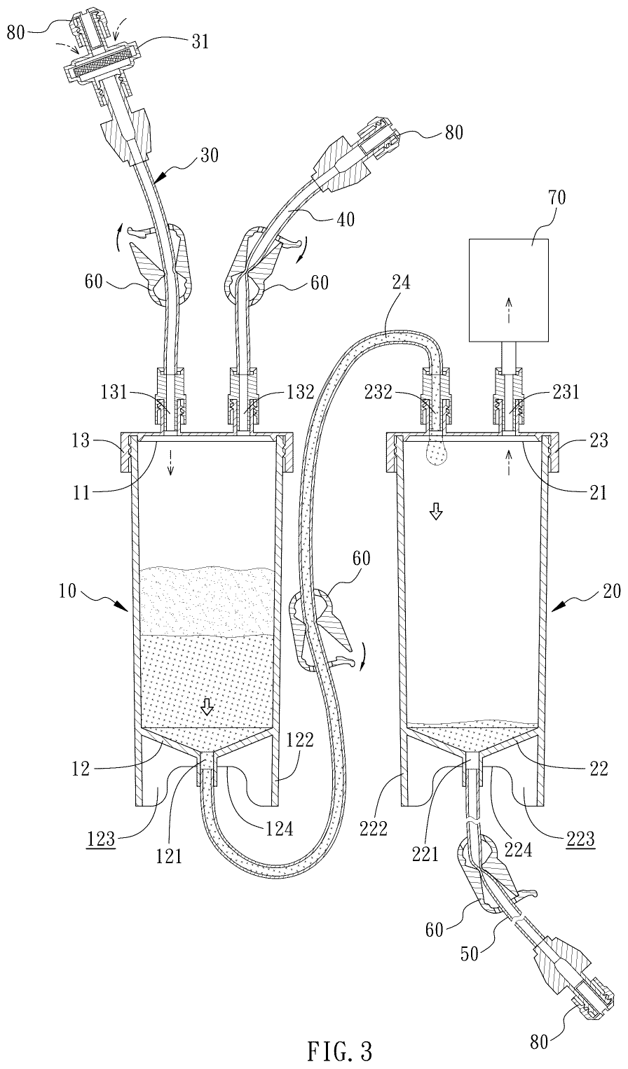

Now referring to FIG. 3, the fluid in the first tube body 10 has separated into a supernatant (e.g., plasma for whole blood centrifugation) and a sediment (e.g., blood cells for whole blood centrifugation). Then, for the sediment to move into the second tube body 20, the valve 60 on the connecting pipe 24 is open and the caps 80 are removed from the venting pipe 30 of the first tube body 10 and the vent 231 of the second tube body 20, respectively. Optionally, an air-pressure source 70 may be removably connected to the vent 231 of the second tube body 20 for accelerated migration of the sediment with a drawing force.

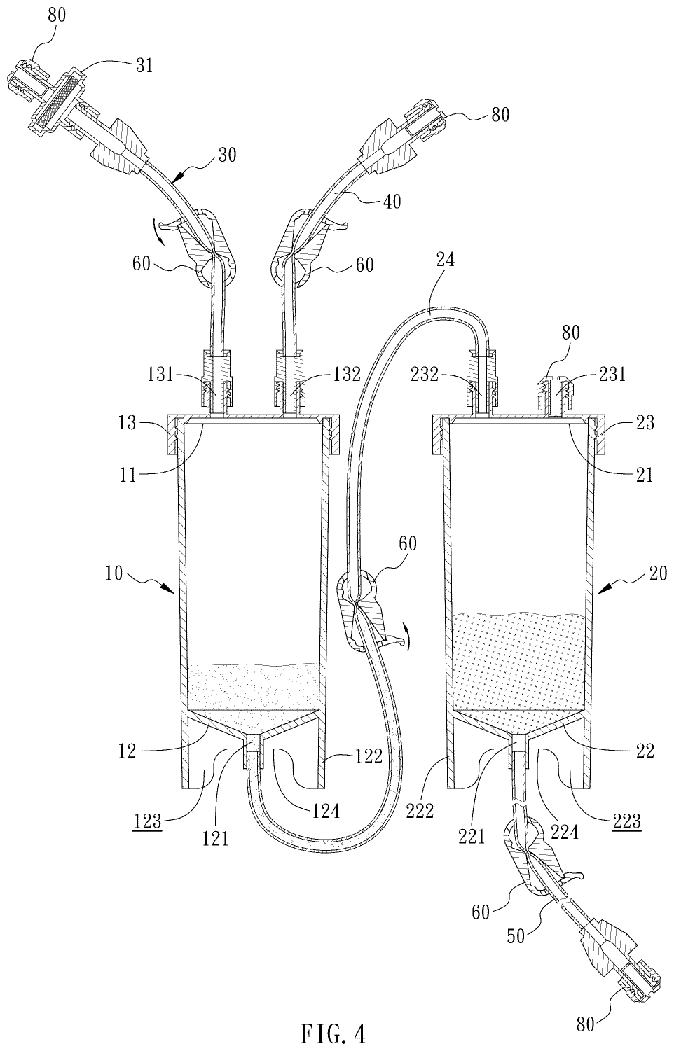

Since the bottom 12 of the first tube body 10 has the funnel-like, or downward conical, structure, and the outlet 121 is at the bottom-most position of the bottom 12 of the first tube body 10, the migration of the sediment can be performed precisely without having the supernatant unintentionally move into the second tube body 20. Besides, as the sediment directly flows into the second tube body 20, the risk of spills can be completely eliminated. After the sediment enters the second tube body 20, the connecting pipe 24 is closed by its valve 60 again, and the vent 231 of the second tube body 20 with the air-pressure source 70 having been detached is capped again, so as to seal the supernatant and the sediment in the first tube body 10 and the second tube body 20, respectively. Consequently, all these enable effective and efficient isolation of the sediment form the supernatant, as shown in FIG. 4.

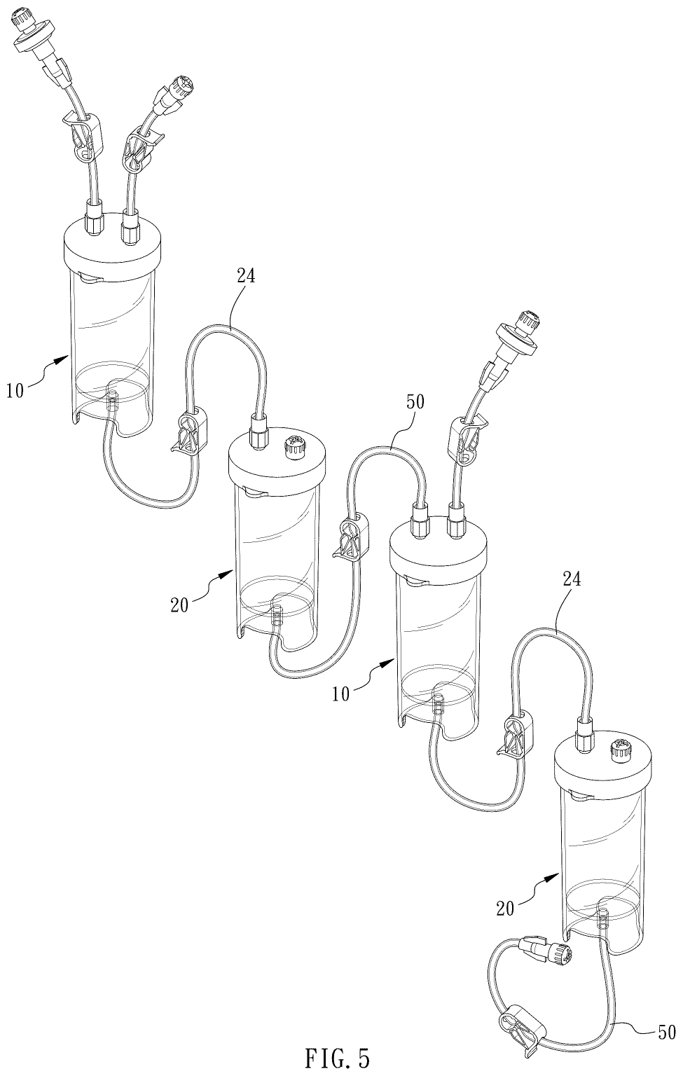

Referring to FIG. 5, for various applications where multiple separations are required, another closed cell separation operation apparatus of the present invention may be connected in series to the first closed cell separation operation apparatus. Specifically, an extension pipe 50 may be used to connect the first tube body 10 of the further closed cell separation operation apparatus. In this case, the extension pipe 50 may be coiled around the outlet 221 and received in the pipe-receiving space 223 of the second tube body 20 during centrifugation as described with respect to the connecting pipe 24.

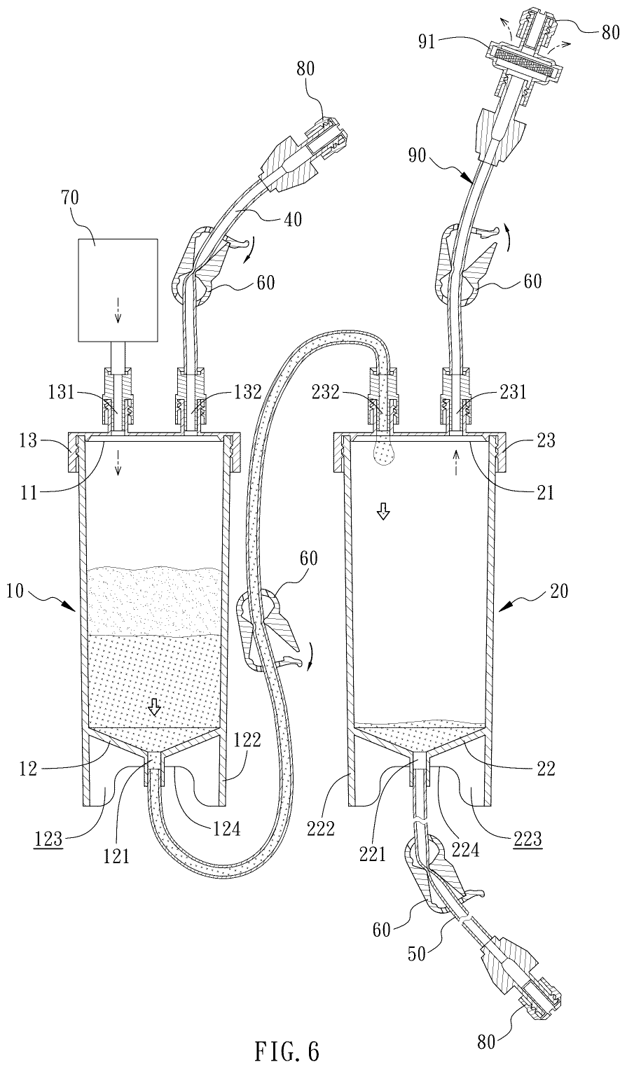

FIG. 6 depicts another embodiment of the closed cell separation operation apparatus of the present invention. As shown in FIG. 6, the closed cell separation operation apparatus is further provided with a second venting pipe 90 attached to the vent 231 of the second tube body 20. The second venting pipe 90 is equipped with a further valve 60 for closing or opening it. The second venting pipe 90 is connected to an additional filter 91 for preventing foreign objects from entering the second tube body 20 and further ensuring a contamination-free operation environment. In this case, an air-pressure source 70, as described previously may be attached to the vent 131 of the first tube body 10 for pushing the content in the first tube body 10 into the second tube body 20.

The foregoing valves 60 may each independently be a tube clamp, a flow control valve, a multi-way connector or an on-off fluid valve, without limitation.

The air-pressure source 70 attached to the vent 131 of the first tube body 10 and/or the vent 231 of the second tube body 20 may be an air pump or an air injector or any other device that can change the pressure in the first tube body 10 and the second tube body 20 to facilitate fluid flow from the first tube body 10 to the second tube body 20.

At last, centrifugation is widely used in various medical and biological applications and well known to those skilled in the art, so the related operation and principle are not described in detail for the sake of succinctness. Likewise, the cell isolation technology involving in the use of the disclosed closed cell separation operation apparatus is also known in the art and not discussed herein.

With the configurations described above, the closed cell separation operation apparatus achieves the objectives of the present invention by providing a closed, contamination-free environment for both centrifugation and post-centrifugation supernatant separation while eliminating the risks of spills and the needs for costly laboratory equipment.

The present invention has been described regarding the preferred embodiments and it is understood that the embodiments are not intended to limit the scope of the present invention. Moreover, all modifications, equivalent changes or improvements which do not depart from the concept of the present invention should be encompassed by the appended claims.

What is claimed is:

1. A closed cell separation operation apparatus, comprising:
a first tube body, being a cylinder-like container defined by a peripheral wall, the first tube body having a closed top that sealably communicates with the exterior through a vent and an inlet, wherein the vent is for allowing air to pass and enter or exit the first tube body and the inlet is for allowing fluid from an external fluid source to enter the first tube body; and the first tube body having a closed bottom that sealably communicates with the exterior through a central outlet;
a second tube body, being a cylinder-like container defined by a peripheral wall, the second tube body having a closed top that sealably communicates with the exterior through a vent and an inlet, wherein the vent is for allowing an air to pass and enter or exit the second tube body and the inlet is for allowing at least one part of the fluid from the outlet of the first tube body to enter the second tube body and the second tube body having a closed bottom that sealably communicates with the exterior through a centrally formed outlet; and
a connecting pipe, being arranged between the outlet of the first tube body and the inlet of the second tube body, so as to link the first tube body and the second tube body.

2. The closed cell separation operation apparatus of claim 1, wherein the peripheral wall of the first tube body extends downward the bottom of the first tube body so the peripheral wall and the bottom form a pipe-receiving space around the outlet of the first tube body.

3. The closed cell separation operation apparatus of claim 2, wherein the peripheral wall of the first tube body has a lower edge formed with a notch for the connecting pipe to pass therethrough.

4. The closed cell separation operation apparatus of claim 1, further comprising an extension pipe attached to the outlet of the second tube body for connecting the second tube body to a further said first tube body of a further said closed cell separation operation apparatus.

5. The closed cell separation operation apparatus of claim 4, wherein the peripheral wall of the second tube body extends downward the bottom of the second tube body so the peripheral wall and the bottom form a pipe-receiving space around the outlet of the second tube body.

6. The closed cell separation operation apparatus of claim 5, wherein the peripheral wall of the second tube body has a lower edge formed with a notch for the connecting pipe to pass therethrough.

7. The closed cell separation operation apparatus of claim 1, wherein the connecting pipe is equipped with a valve for closing or opening the connecting pipe.

8. The closed cell separation operation apparatus of claim 4, wherein the extension pipe is equipped with a valve for closing or opening the extension pipe.

9. The closed cell separation operation apparatus of claim 2, wherein the bottom of the first tube body has a downward conical structure with a diameter gradually reduces and reaches a smallest extent thereof at a periphery of the outlet of the first tube body.

10. The closed cell separation operation apparatus of claim 5, wherein the bottom of the second tube body has a downward conical structure with a diameter gradually reduces and reaches a smallest extent thereof at a periphery of the outlet of the second tube body.

11. The closed cell separation operation apparatus of claim 1, wherein at least one of the vents of the first tube body and the second tube body is equipped with a filter for filtering the air passing through the vent.

12. The closed cell separation operation apparatus of claim 1, wherein at least one of the vents of the first tube body and the second tube body is equipped with an air-pressure source for pushing an air into or drawing an air from the first tube body and the second tube body.

13. The closed cell separation operation apparatus of claim 1, wherein at least one of the inlets and the vents of the first tube body and the second tube body is configured to be detachably sealed by a seal in an airtight manner.

14. The closed cell separation operation apparatus of claim 1, wherein at least one of the inlets and the vents of the first tube body and the second tube body is further provided with a pipe that is equipped with a vale.
